(19) United States
(12) Patent Application Publication
(10) Pub. No.: US 2004/0267313 A1
(43) Pub. Date: Dec. 30, 2004

(54) High strength multi-component surgical cord

(75) Inventors: Drew Amery, Clearwater, FL (US); Amber Powell, St. Petersburg, FL (US)

Correspondence Address: H. JAY SPIEGEL, P. O. BOX 11, Mount Vernon, VA, 22121, US

(73) Assignee: LINVATEC CORPORATION

(21) Appl. No.: 10/874,301

(22) Filed: Jun. 24, 2004

Related U.S. Application Data
(60) Provisional application No. 60/483,084, filed on Jun. 27, 2003.

(51) Int. Cl. A61L 17/00; A61B 17/04

(52) U.S. Cl. 606/228

(57) ABSTRACT

In the present invention, a gel-spun UHMWPE is subjected to secondary processing to reduce the concentration of the spin solvent(s) to acceptable levels before or after the fiber is woven with other fibers to form a suture. In the case of the spin solvent Decalin, it is well known that concentrations, in the fiber, of greater than 100 parts per million (ppm) render UHMWPE materials non-compatible with human tissues. Thus, UHMWPE materials that are to remain within the human body must have a Decalin concentration below 100 ppm to render them biocompatible. If desired, the suture may be braided about a core. The number of filaments in a single suture can vary between 4 and 24. Additionally, where a core structure is employed, up to 6 separate core filaments may be employed. Other materials such as PTFE, FEP, PFA, PVFD, PP, polyester, nylon, or aramid may be employed for the core and/or cover so long as spin solvent level has been reduced to below 100 ppm before or after twisting or braiding.

### BACKGROUND OF THE INVENTION

1. Field of The Invention

The invention relates to a braided suture and a process for producing a braided suture. More specifically, it refers to a braided biocompatible suture made from ultra high molecular weight polyethylene filaments.

2. Background of the Prior Art

Braided multifilament sutures are commonly used in surgery because of their excellent combination of properties. These property requirements include biocompatibility, sterilizability, tensile strength, diameter, slidability, and knot-tying retention properties.

Surgical sutures are commonly made from a variety of synthetic melt spun polymer materials including polyesters (e.g. polyethylene terephthalate, polybutyl terephthalate), polyolefins (e.g. polypropylene and polyethylene), fluoropolymers (e.g. polytetrafluoroethylene, polyvinylidene fluoride), polyamide (e.g. nylon, nylon 6, nylon 6-6), or copolymers and blends.

Ultra High Molecular Weight Polyethylene (UHMWPE) has been used as a hip implant component for over twenty years with excellent biological response and highly lubricous surface. Because of its high molecular weight, conventional melt processing is extremely difficult. Traditionally, powdered metallurgy sintering processes have been utilized to manufacture 3-Dimensional shapes. A gel-spinning process is described in U.S. Pat. No. 4,413,110 (High Tenacity, High Modulus Polyethylene and Polypropylene Fibers and Intermediates Therefore) to produce oriented UHMWPE fibers with tensile properties vastly superior to un-oriented polyethylene. Oriented UHMWPE fibers are sold under the trade names of Spectra™ and Dyneema™. During the production process, gel spinning solvent (typically Decalin, CASRN 91-7-8)is entrapped in the filament. The concentration of Decalin is uncontrolled and typically greater than 1000 parts-per-million (ppm). Decalin is a material known to be an irritant to the skin, eyes and mucous membranes on acute exposure. The lowest published effective dose for producing irritation in humans is 100 ppm. (toxnet.nlm.nih.gov)

Composite surgical sutures are described in U.S. Pat. No. 4,470,941 (Preparation of Composite Surgical Sutures). This disclosure describes a composite suture comprising a core of low-melting fibers around which are braided high-melting fibers. Because of the difference in melting temperatures, when subjected to heat, the low melting core redistributes throughout the braid of high melting fibers. Although this composite combines the best properties of two different fiber components, its increased stiffness and loss of molecular orientation of the core results in a product that resembles monofilament fiber.

Sutures based on heterogeneous braids are also described in U.S. Pat. No. 5,314,446 (Sterilized Heterogeneous Braids). This disclosure specifically attempts to improve the fiber-fiber friction and its impact on fiber mobility and braid pliability without appreciably sacrificing its physical properties. While the focus of the disclosure is specifically melt spun PET and PTFE composites, other polymers with surface energies less than 30 dynes/cm are listed. The second components described are fibers manufactured from melt spun yarns of PET, nylon, or aramid with a yarn tenacity greater than 3.0 grams/denier.

| Component 1 | Component 2 |
|---|---|
| Specification: | Specification: |
| Surface Energy <30 dynes/cn | Yarn tenacity >3.0 grams/denier |
| Polytetrafluoroethylene (PTFE) | PET |
| Ethylene/propylene compolymers | Nylon |
| (FEP) | |
| Perfluoroalkoxy (PFA) polymers | Aramid |
| Polyvinylidene Fluoride (PVDF) | |
| Polyethylene/tetrafluorethylene | |
| copolymers (PETFE) | |
| Polychlorofluorethylene (PCFE) | |
| Polypropylene (PP) | |
| Polyethylene (PE) | |

This disclosure focuses on the use of “lubricating yarns” to improve the pliability without appreciably sacrificing its physical properties.

U.S. Pat. No. 5,318,575 describes the use of ultra-high molecular weight high tenacity polyethylene fibers combined with PET or nylon to manufacture sternum closure devices, specifically surgical tapes. The described preferred embodiment had a tensile strength greater or equal to 35 kg straight pull (77.16 lbf) combined with elongation at break of less than 15%. While this prior art combines the use of UHMWPE for increased construct strength, the utilization of materials that are biologically compatible is not discussed or implied.

The attempts described in the prior art to improve braid properties have overlooked the importance of biocompatibility caused by the method of fiber manufacture. Such biocompatibility needs to be developed.

### SUMMARY OF THE INVENTION

The present invention provides a high strength suture manufactured with UHMWPE and PET fibers that offer increased strength combined with knot-tying, knot retention, and suitable biocompatibility.

(1) The present invention contemplates employing a gel-spun UHMWPE, in which, as is customary, a gel spinning solvent such as, for example, Decalin has been employed during the process of manufacture. However, in order to facilitate the biocompatibility of a suture manufactured from such a fiber, secondary processing is undertaken to reduce the concentration of the spin solvent(s) to acceptable levels before the fiber is woven with other fibers to form a suture.

(2) In the case of Decalin, it is well known that concentrations, in the fiber, of greater than 100 parts per million (ppm) render UHMWPE materials non-compatible with human tissues. Thus, UHMWPE materials that are to remain within the human body must have a Decalin concentration below 100 ppm to render them biocompatible.

(3) Once the UHMWPE fibers have been secondarily processed so that the concentration of spin solvent(s) has been reduced to a level rendering the material biocompatible, thereafter, such fibers are conventionally braided to create a suture.

(4) If desired, the suture may be braided about a core or, alternatively, the core may be omitted. Various manners of braiding may be employed such as, for example, spiral or lattice weaving.

(5) In the preferred embodiments of the present invention, the number of filaments in a single suture can vary between 4 and 24. Additionally, where a core structure is employed, up to 6 separate core filaments may be employed.

(6) If desired, the suture can be coated with a suitable biocompatible substance such as, for example, silicone. In use, as is well known, the suture may be attached to a needle and/or a soft tissue anchor.

(7) Other materials may be employed for the core and/or cover so long as spin solvent level has been reduced to below 100 ppm before or after twisting or braiding. Such materials include PTFE, FEP, PFA, PVFD, PP, polyester, nylon, or aramid, and may, if desired, be intertwined with filaments or fibers of UHMWPE.

As such, it is a first object of the present invention to provide a biocompatible suture made of ultra high molecular weight polyethylene (UHMWPE).

It is a further object of the present invention to provide such a suture in which fibers of UHMWPE have been secondarily treated, after their initial manufacture, to reduce the concentration of non-biocompatible solvent(s) to a level rendering the fiber biocompatible.

It is a still further object of the present invention to provide such a suture in which such secondarily treated UHMWPE fibers are woven to create a suture.

It is a still further object of the present invention to provide such a suture in which the UHMWPE fibers are woven or braided about a core consisting of one or more fibers.

It is a yet further object of the present invention to provide such a suture in which 4 to 24 fibers are woven or braided to form the suture.

It is a still further object of the present invention to provide such a suture in which a core, if employed, includes from 1 to 6 fibers.

These and other objects, aspects and features of the present invention will be better understood from the following detailed description of the preferred embodiments when read in conjunction with the single drawing figure.

### BRIEF DESCRIPTION OF THE DRAWINGS

Various embodiments of the present invention will be discussed with reference to the appended drawing. This drawing depicts only an illustrative embodiment of the invention and is not to be considered limiting of its scope.

FIG. 1 is a cross-sectional view of an eight yarn braid with core.

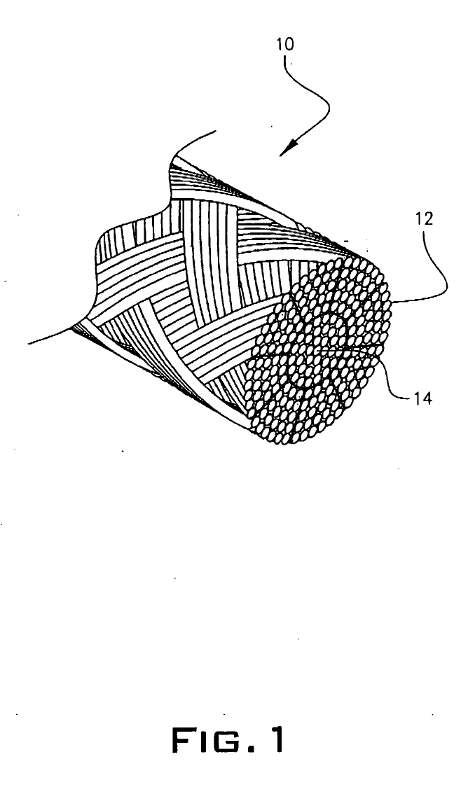

### DETAILED DESCRIPTION OF THE INVENTION

Regular UHMWPE has a history of safe and effective use for replacement hip applications. To improve the biocompatibility of sutures manufactured from gel-spun UHMWPE, the present invention utilizes gel-spun UHMWPE that has undergone secondary processing to reduce the spin solvent(s) to acceptable levels before braid manufacture; in the case of Decalin, concentrations less than the published level of irritation in humans (<100 ppm). The biocompatible UHMWPE fibers suitable for use in the present invention are marketed under the Trademark Dyneema Purity™ by DSM of the Netherlands. The exact method of purification is a trade secret of DSM.

The braids 12 of this invention are conventionally braided in a tubular sheath 10 around a core 14 of fibers, although such a core may be excluded if desired. U.S. Pat. Nos. 3,187,752; 4,043,344; and 4,047,533 are examples of braid structures with cores and are incorporated herein by reference. Alternatively, the braids could be woven in a spiral or lattices as described in U.S. Pat. Nos. 4,959,069 and 5,059,213, incorporated herein by reference.

While not totally inclusive, some of the preferred embodiments include:

A surgical braid comprising a cover and core, the cover manufactured from multifilament fibers of biocompatible UHMWPE and/or biocompatible UHMWPE composite fibers. The number of filaments in braid 12 can vary between 4 and 24. The core 14 consists of twisted fibers of biocompatible UHMWPE and/or biocompatible UHMWPE composite fibers. The core 14 also can consist of other polymers known to the art. The number of core 14 filaments can vary from 0 to 6.

In the following examples, the tensile properties and knot security were determined using a Tinius Olsen Tensile Tester using the methodology described in the US Pharmacopoeia 24. The examples are only illustrative and not intended to limit the scope of the claimed invention.

### EXAMPLES

Control: Ethibond Polyester Suture: Commercially available. Size USP #2.

| | | | | Knot |
|---|---|---|---|---|
| | Composition braid | | Diameter | Strength |
| ID | PET:UHMWPE | Composition Core | (mm) | (lbf) |
| 1 | 100:0, 16 carrier | 4 ply low twist | 0.50 | 43.12 |
| | 48 ppi | UHMWPE | | |
| 2 | 100:0, 16 carrier | 2 ply PET | 0.52 | 17.84 |
| | 48 ppi | | | |
| 3 | 50:50, 16 carrier | 5 ply low twist | 0.58 | 33.34 |
| | 48 ppi | UHMWPE | | |
| 4 | 100:0, 16 carrier | 6 ply low twist | 0.53 | 21.89 |
| | 48 ppi | UHMWPE | | |
| 5 | 50:50, 16 carrier | 2 ply low twist PET | 0.52 | 27.15 |
| | 48 ppi | | | |
| 6 | 50:50, 16 carrier | 6 ply high twist | 0.58 | 36.94 |
| | 48 ppi | UHMWPE | | |
| 7 | 100:0, 16 carrier | 6 ply high twist | 0.52 | 20.11 |
| | 48 ppi | UHMWPE | | |
| 8 | 50:50, 16 carrier | 6 ply twisted PET | 0.55 | 26.03 |
| | 48 ppi | | | |
| 9 | #2 Ethibond PET | PET | N/A | 15.31 |

If desired, the suture can be coated with a suitable biocompatible substance. In use, as is well known, the suture may be attached to a needle and/or a soft tissue anchor.

Other materials may be employed for the core and/or cover so long as spin solvent level has been reduced to below 100 ppm before or after twisting or braiding. Such materials include PTFE, FEP, PFA, PVFD, PP, polyester, nylon, or aramid, and may, if desired, be intertwined with filaments or fibers of UHMWPE.

As such, an invention has been disclosed in terms of preferred embodiments thereof which fulfill each and every one of the objects of the invention as set forth hereinabove, and provide a new and useful high strength multi-component surgical cord of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. A biocompatible surgical suture containing fibers of ultra high molecular weight polyethylene (UHMWPE), the suture comprising:
a core of 0 to 6 twisted biocompatible polymer filaments and a cover of multiple filaments of braided gel-spun UHMWPE, such UHMWPE filaments having undergone secondary processing to reduce spin solvent level to below 100 ppm before braiding.

2. The suture of claim 1, wherein said core filaments are made of UHMWPE.

3. The suture of claim 2, wherein said core filaments, before twisting, undergo secondary processing to reduce spin solvent level to below 100 ppm.

4. The suture of claim 1, provided with a protective coating.

5. The suture of claim 1, attached to a soft tissue anchor.

6. A biocompatible surgical suture containing fibers of ultra high molecular weight polyethylene (UHMWPE), the suture comprising:
a core of 0 to 6 twisted biocompatible polymer filaments and a cover of multiple filaments of braided gel-spun UHMWPE, such UHMWPE filaments having undergone secondary processing to reduce spin solvent level to below 100 ppm after braiding.

7. The suture of claim 6, wherein said core filaments are made of UHMWPE.

8. The suture of claim 7, wherein said core filaments, after twisting, undergo secondary processing to reduce spin solvent level to below 100 ppm.

9. The suture of claim 6, provided with a protective coating.

10. The suture of claim 6, attached to a soft tissue anchor.

11. A biocompatible surgical suture containing fibers of ultra high molecular weight polyethylene (UHMWPE), the suture comprising:
a core of twisted biocompatible polymer filaments including one or more first core filaments in intertwining contact with one or more second core filaments, said second core filaments being made of a material selected from the group consisting of PTFE, FEP, PFA, PVDF, PP, polyester, nylon and aramid, and a cover of multiple filaments including one or more first cover filaments in intertwining contact with one or more second cover filaments, said first cover filaments made of UHMWPE and said second cover filaments being made of a material selected from the group consisting of PTFE, FEP, PFA, PVDF, PP, polyester, nylon and aramid, such cover filaments having undergone secondary processing to reduce spin solvent level to below 100 ppm before braiding.

12. The suture of claim 1, wherein said first core filaments are made of UHMWPE.

13. The suture of claim 12, wherein said core filaments, before twisting, undergo secondary processing to reduce spin solvent level to below 100 ppm.

14. The suture of claim 11, provided with a protective coating.

15. The suture of claim 11, attached to a soft tissue anchor.

16. A biocompatible surgical suture containing fibers of ultra high molecular weight polyethylene (UHMWPE), the suture comprising:
a core of twisted biocompatible polymer filaments including one or more first core filaments in intertwining contact with one or more second core filaments, said second core filaments being made of a material selected from the group consisting of PTFE, FEP, PFA, PVDF, PP, polyester, nylon and aramid, and a cover of multiple filaments including one or more first cover filaments in intertwining contact with one or more second cover filaments, said first cover filaments made of UHMWPE and said second cover filaments being made of a material selected from the group consisting of PTFE, FEP, PFA, PVDF, PP, polyester, nylon and aramid, such cover filaments having undergone secondary processing to reduce spin solvent level to below 100 ppm after braiding.

17. The suture of claim 16, wherein said first core filaments are made of UHMWPE.

18. The suture of claim 17, wherein said core filaments, after twisting, undergo secondary processing to reduce spin solvent level to below 100 ppm.

19. The suture of claim 16, provided with a protective coating.

20. The suture of claim 16, attached to a soft tissue anchor.
